(19) United States
(12) Patent Application Publication
(10) Pub. No.: US 2020/0345958 A1
(43) Pub. Date: Nov. 5, 2020

(54) FLUID PRODUCT DISPENSING DEVICE

(71) Applicant: APTAR FRANCE SAS, LE NEUBOURG (FR)

(72) Inventor: Patrice LEONE, ACQUIGNY (FR)

(73) Assignee: APTAR FRANCE SAS, LE NEUBOURG (FR)

(21) Appl. No.: 16/961,961

(22) PCT Filed: Jan. 24, 2019
(86) PCT No.: PCT/FR2019/000011

§ 371 Date: Jul. 14, 2020

(30) Foreign Application Priority Data
Jan. 24, 2018 (FR) 1850540

(51) Int. Cl. A61M 15/00; C08L 73/00; B05B 11/00

(52) U.S. Cl. CPC A61M 15/0073; C08L 73/00; A61M 2205/0222; B05B 11/3053; B05B 11/308

(57) ABSTRACT

A fluid dispenser device having at least one reservoir (20) that is suitable for containing fluid to be dispensed, a dispenser member (1, 30), an actuator member (2, 10) that is suitable for actuating said dispenser member (1, 30); a dispenser orifice (3, 40) via which the fluid is dispensed; and a dose counter or indicator (A). At least a portion of the dose counter or indicator (A) is made out of a material having an aliphatic polyketone.

The present invention relates to a fluid dispenser device.

The preferred field of application for such a dispenser device is, particularly, but not exclusively, the field of pharmacy.

Fluid dispensers of the prior art generally include a dispenser member, such as a pump or a valve, that is in communication firstly with one or more fluid reservoirs, and secondly with an actuator member for actuating said dispenser member. Some dispensers may also include a dose counter or indicator for indicating to the user the number of doses that have been dispensed or that remain to be dispensed. Portions of such counter devices are often made out of plastics materials, in particular such as polybutyl terephthalate (PBT), polyoxymethylene (POM), polyamide (PA), or polyethylene (PE). Unfortunately, the use of such plastics materials can result in various drawbacks. Thus, coefficients of friction, in particular for POM, are not good, and can lead to the device malfunctioning. In addition, mechanical properties, in particular for POM, are not good, and can also have a negative impact on the actuation of the device. In addition, production costs, in particular for devices using the materials listed above, can turn out to be high, in particular as a result of cycle times that can be quite long.

An object of the present invention is to overcome the above-mentioned problems.

An object of the present invention is thus to provide a dispenser device that enables counting in reliable and reproducible manner on each actuation.

Another object of the present invention is to provide a fluid dispenser device that is simple and inexpensive to manufacture.

^{†} Translation of the title as established ex officio.

The present invention thus provides a fluid dispenser device comprising:
at least one reservoir that is suitable for containing fluid to be dispensed;
a dispenser member;
an actuator member that is suitable for actuating said dispenser member;
a dispenser orifice via which the fluid is dispensed; and
a dose counter or indicator;

at least a portion of said dose counter or indicator being made out of a material comprising an aliphatic polyketone.

Advantageously, said material comprises an aliphatic polyketone terpolymer.

Advantageously, said terpolymer is an ethylene/propylene/carbon monoxide terpolymer, having the formula:

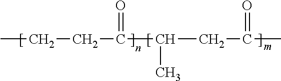

In a first advantageous embodiment, said dispenser member is a valve, in particular a metering valve containing a propellant gas for dispensing the fluid.

Advantageously, the device contains a hydrofluoroalkane (HFA) gas as a propellant gas.

In a second advantageous embodiment, said dispenser member is a pump.

Advantageously, said material is constituted by aliphatic polyketone.

In a variant, said material is an alloy comprising at least one aliphatic polyketone and at least one other polymer.

Advantageously, said at least one other polymer comprises one or more of the following polymers: polymethyl methacrylate (PMMA), polybutyl terephthalate (PBT), polyacetal (POM), polyethylene glycol (PETG), polyvinyl chloride (PVC), polyamide (PA), polycarbonate (PC), polystyrene (PS), styrene acrylonitrile (SAN), acrylonitrile butadiene styrene (ABS), high-density polyethylene (HDPE), low-density polyethylene (LDPE), polysulfone (PSU) alloy, polyethylene terephthalate (PET), thermoplastic polyurethane (TPUR) elastomer, polyphenylene sulfide (PPS), polyethersulfone (PES), thermoplastic polyester elastomer (TPE), modified polyphenylene oxide (PPO), polyetherimide (PEI), polyetheretherketone (PEEK), rigid thermoplastic polyurethane (RTPU), saturated styrenic elastomer (SEBS), unsaturated styrenic elastomer (SBS), olefinic thermoplastic elastomer (TEO), vulcanized styrenic elastomer (TPV), polymethylpentene (PMP), perfluoroalkoxy (PFA), ethylene tetrafluoroethylene (ETFE), polyvinylidene fluoride (PVDF), liquid crystal polymer (LCP), fluorinated ethylene propylene (FEP), polyphtalamide (PPA), polyetherketoneketone (PEKK), thermoplastic polyimide (TPI), high-temperature polyamide (NHT), syndiotactic polystyrene (SPS), polytrimethylene terephthalate (PTT).

These and other characteristics and advantages of the present invention appear more clearly from the following detailed description, given by way of non-limiting examples, and with reference to the accompanying drawings, and in which:

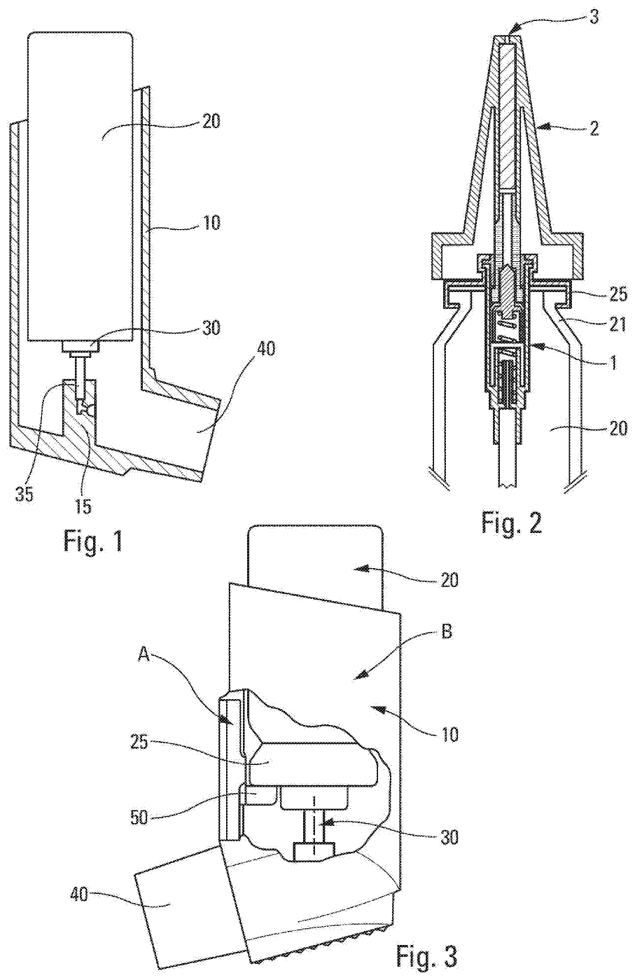

FIG. 1 shows an example of a dispenser device;

FIG. 2 shows another example of a dispenser device;

FIG. 3 shows an example of a dispenser device to which the present invention applies;

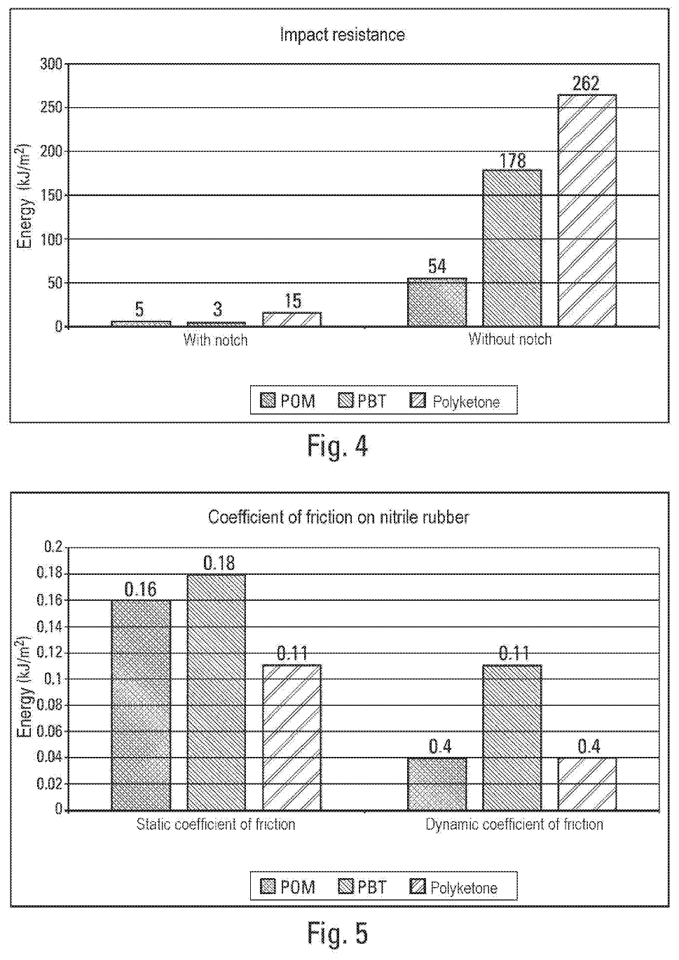

FIG. 4 shows the impact resistance or “toughness” of three different materials;

FIG. 5 shows the coefficient of friction of three different materials on nitrile rubber; and

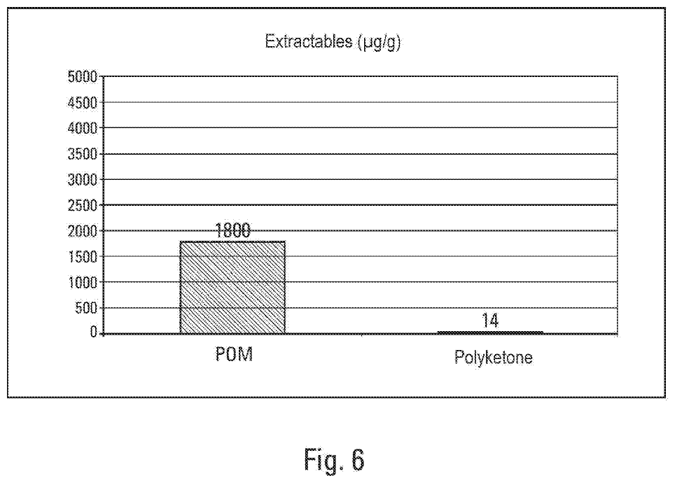

FIG. 6 shows the levels of extractables for two different materials.

The present invention applies to fluid dispenser devices of all types that include a dose counter device. In particular, the present invention may apply to counter dispensers that have a valve, as shown in FIG. 1, or a pump, as shown in FIG. 2.

With reference to FIG. 1, there is described a pressurized metered dose inhaler, generally known as a pMDI, that conventionally includes a body 10 provided with a dispenser orifice 40, generally a mouthpiece. Inside the body there is disposed a reservoir 20 on which a metering valve 30 is mounted. A valve member 35 slides in the valve body of said metering valve 30 so as to dispense a dose of fluid on each actuation. The body 10 includes a well 15 that receives the valve member 35, and that creates a connection passage between the outlet of the valve member 35 and said dispenser orifice 40. In conventional manner, in order to actuate such a device, the user presses on the end of the reservoir 20 so as to push said reservoir axially inside the body 10, thereby causing the valve member 35 to slide in leaktight manner into the metering valve, thereby causing a dose of fluid to be dispensed. Inside the reservoir, the fluid, which generally contains one or more active substances, is associated with a propellant gas, preferably a gas of the HFA type, e.g. HFA 134a and/or HFA 227 and/or HFA 152a. Generally, there is provided in the reservoir 20, around the body of the metering valve 30, a ring known as a can end (not shown), in particular so as to limit the dead volume in the reservoir.

Typically, in devices of this type, valve bodies are made out of PBT or POM, the valve member is made out of POM or PBT, and the ring is made out of PA or PE.

With reference to FIG. 2, the dispenser device comprises a pump 1 that is fastened on the neck 21 of a container 20, e.g. by means of a fastener ring 25. The device further comprises a dispenser head 2. The dispenser head 2 includes an expulsion channel that connects the pump 1 to a fluid dispenser orifice 3. Advantageously, in the embodiment shown in FIG. 2, the dispenser head 2 is a nasal pusher. Naturally, the invention applies to any other type of pump and/or pusher.

Typically, in devices of this type, valve bodies are made out of PBT or POM, and the piston is made out of POM.

FIG. 3 is a diagram showing a dispenser device B provided with a dose indicator A. The device B comprises a body 10 and a reservoir 20 on which a metering valve 30 is assembled by means of a fastener ring 25, such as a crimping cap. The device B is actuated by moving the reservoir 20 axially inside the body 10, such movement causing the valve member of the valve 30 to compress, and this causes a dose of fluid to be expelled through a mouthpiece 40, and also causes the dose indicator A to be actuated by an actuator 50 moving axially. Naturally, this is only one embodiment, and the present invention could be adapted to dose counters or indicators of any type, regardless of whether they are associated with valves or with pumps.

Typically, in this type of dose counter or indicator device, the actuator is generally made out of POM.

In the invention, at least a portion of the dose counter or indicator A is made out of a material comprising an aliphatic polyketone.

Polyketones are high performance thermoplastics having the following formula:

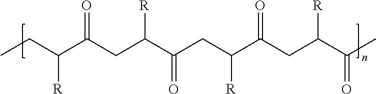

Polyketones are divided into two families: aliphatic polyketones, also knowns as POK, and aromatic polyketones, such as polyetheretherketone, more commonly known as PEEK.

Aliphatic polyketones appeared in the 1990s then disappeared in 2000 as a result of being difficult to work. The Korean company Hyosung relaunched them in 2013. In particular, it has developed terpolymers (ethylene, propylene, copolymer) that can have better processability or workability (in particular a melting temperature that is lower):

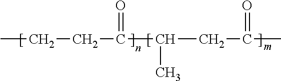

### Ethylene/Propylene/Copolymer

The presence of carbonyl groups in the main chain of their chemical structure imparts advantageous properties thereto, such as:
good wear resistance;
good resistance to hydrolysis;
high level mechanical properties; and
short molding-cycle times.

The usual applications of polyketones are in the oil industry. In particular, it may be advantageous to use them in order to limit the migration of chemical substances in transport systems, or in order to limit corrosion in such transport systems. In the automobile industry, they may be used for connectors where it is necessary to withstand high temperatures and have the ability to withstand fuel. In construction, in climates with high temperatures, nylon filled with glass fibers can advantageously be replaced by polyketones filled with glass fibers.

Polyketones, in particular aliphatic polyketones, have never been used as material for making portions of a dose counter or indicator.

For this type of application, it is not necessary to have good sealing against fuels, nor the ability to withstand high temperatures. In contrast, the mechanical strength of polyketones and the low friction turn out to be advantageous for counter components.

Compared to the materials usually used, as listed above, aliphatic polyketones present in particular the following advantages:
resolution of the formaldehyde problem associated with POM;
improved coefficients of friction compared to PBT;
better mechanical properties than PA; and
savings in production costs as a result of shorter cycle times.

### Mechanical Properties:

One of the tests for characterizing the mechanical properties of a material consists in measuring its impact resistance or “toughness”. The principle consists in determining the energy needed to fracture, in a single impact, a sample that has optionally been notched beforehand. The energy needed for fracture to occur is obtained by calculating the potential difference for the hammer between its start position (highest position) and its end position after the sample has been fractured.

In the FIG. 4 bar chart, a clear improvement can be observed in the impact resistance of polyketone, compared to PBT and POM.

### Friction:

The test consists in rubbing two materials together so as to determine their coefficient of friction. The material used to perform this comparative test was nitrile rubber.

The coefficient of friction is the ratio of the traction force (response force enabling the apparatus to move) over the applied force (normal force).

Two types of coefficient of friction exist: a coefficient of dynamic friction and a coefficient of static friction.
The static coefficient of friction is the coefficient measured at the beginning of a test; it is the force necessary to move the sample on the substrate and to initiate movement; the term “coefficient of adhesion” is also used;
The dynamic coefficient of friction is the coefficient that is otherwise necessary for movement to be maintained at a constant speed.

The results obtained, plotted in FIG. 5, show that the aliphatic polyketones have:
a static coefficient of friction that is less than PBT and POM; and
a dynamic coefficient of friction that is less than PBT and at the same level as POM.

### Extractables:

FIG. 6 shows that the level of extractables measured for aliphatic polyketones are much less than the level of extractables of POM.

It is possible to make the portions of the dose counter or indicator from a material comprising an alloy of at least one aliphatic polyketone and at least one other polymer. In order to form such alloys, said at least one other polymer can be selected from the following polymers: polymethyl methacrylate (PMMA), polybutyl terephthalate (PBT), polyacetal (POM), polyethylene glycol (PETG), polyvinyl chloride (PVC), polyamide (PA), polycarbonate (PC), polystyrene (PS), styrene acrylonitrile (SAN), acrylonitrile butadiene styrene (ABS), high-density polyethylene (HDPE), low-density polyethylene (LDPE), polysulfone (PSU) alloy, polyethylene terephthalate (PET), thermoplastic polyurethane (TPUR) elastomer, polyphenylene sulfide (PPS), polyethersulfone (PES), thermoplastic polyester elastomer (TPE), modified polyphenylene oxide (PPO), polyetherimide (PEI), polyetheretherketone (PEEK), rigid thermoplastic polyurethane (RTPU), saturated styrenic elastomer (SEBS), unsaturated styrenic elastomer (SBS), olefinic thermoplastic elastomer (TEO), vulcanized styrenic elastomer (TPV), polymethylpentene (PMP), perfluoroalkoxy (PFA), ethylene tetrafluoroethylene (ETFE), polyvinylidene fluoride (PVDF), liquid crystal polymer (LCP), fluorinated ethylene propylene (FEP), polyphtalamide (PPA), polyetherketoneketone (PEKK), thermoplastic polyimide (TPI), high-temperature polyamide (NHT), syndiotactic polystyrene (SPS), polytrimethylene terephthalate (PTT). However, this list of polymers should not be considered as being limiting, any polymer that is suitable for being combined with said at least one aliphatic polyketone can be used.

Consequently, the present invention proposes an advantageous and effective solution for optimizing the properties of the material. The invention is thus, particularly, but not exclusively, advantageous specifically for dispensing pharmaceutical formulations.

The present invention is described above with reference to several advantageous embodiments, but naturally any modification could be applied thereto by a person skilled in the art, without going beyond the ambit of the present invention, as defined by the accompanying claims.

1. A fluid dispenser device comprising:
at least one reservoir (20) that is suitable for containing fluid to be dispensed;
a dispenser member (1; 30);
an actuator member (2; 10) that is suitable for actuating said dispenser member (1; 30);
a dispenser orifice (3; 40) via which the fluid is dispensed; and
a dose counter or indicator (A);
said dispenser device being characterized in that at least a portion of said dose counter or indicator (A) is made out of a material comprising an aliphatic polyketone.

2. A device according to claim 1, wherein said material comprises an aliphatic polyketone terpolymer.

3. A device according to claim 2, wherein said terpolymer is an ethylene/propylene/carbon monoxide terpolymer, having the formula:

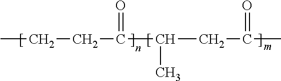

4. A device according to claim 1, wherein said dispenser member is a valve (30), in particular a metering valve containing a propellant gas for dispensing the fluid.

5. A device according to claim 4, containing an HFA gas as a propellant gas.

6. A device according to claim 1, wherein
said dispenser member is a pump (1).

7. A device according to claim 1, wherein said material is constituted by aliphatic polyketone.

8. A device according to claim 1, wherein said material is an alloy comprising at least one aliphatic polyketone and at least one other polymer.

9. A device according to claim 8, wherein said at least one other polymer comprises one or more of the following polymers: polymethyl methacrylate (PMMA), polybutyl terephthalate (PBT), polyacetal (POM), polyethylene glycol (PETG), polyvinyl chloride (PVC), polyamide (PA), polycarbonate (PC), polystyrene (PS), styrene acrylonitrile (SAN), acrylonitrile butadiene styrene (ABS), high-density polyethylene (HDPE), low-density polyethylene (LDPE), polysulfone (PSU) alloy, polyethylene terephthalate (PET), thermoplastic polyurethane (TPUR) elastomer, polyphenylene sulfide (PPS), polyethersulfone (PES), thermoplastic polyester elastomer (TPE), modified polyphenylene oxide (PPO), polyetherimide (PEI), polyetheretherketone (PEEK), rigid thermoplastic polyurethane (RTPU), saturated styrenic elastomer (SEBS), unsaturated styrenic elastomer (SBS), olefinic thermoplastic elastomer (TEO), vulcanized styrenic elastomer (TPV), polymethylpentene (PMP), perfluoroalkoxy (PFA), ethylene tetrafluoroethylene (ETFE), polyvinylidene fluoride (PVDF), liquid crystal polymer (LCP), fluorinated ethylene propylene (FEP), polyphtalamide (PPA), polyetherketoneketone (PEKK), thermoplastic polyimide (TPI), high-temperature polyamide (NHT), syndiotactic polystyrene (SPS), polytrimethylene terephthalate (PTT).
